(19) United States
(12) Patent Application Publication
(10) Pub. No.: US 2002/0037319 A1
(43) Pub. Date: Mar. 28, 2002

(54) Drug preparations

(76) Inventors: Alan Drizen, Downsview (CA); Michael Micalizzi, Miami, FL (US)

Correspondence Address: NATH & ASSOCIATES, 1030 15th STREET, 6TH FLOOR, WASHINGTON, DC, 20005, US

(21) Appl. No.: 09/849,401

(22) Filed: May 7, 2001

Related U.S. Application Data
(63) Continuation-in-part of application No. 09/708,069, filed on Nov. 8, 2000.
(60) Provisional application No. 60/164,149, filed on Nov. 8, 1999.

(51) Int. Cl. A61K 9/14; A61K 31/715

(52) U.S. Cl. 424/488; 514/54

(57) ABSTRACT

Semisolid, sustained-release drug delivery compositions based on hyaluronic acid and its salts, and more particularly to the manufacture and use of such compositions.

### FIELD OF THE INVENTION

This invention relates to the preparation of a transdermal delivery system. The preparation is designed to deliver therapeutic levels of a drug to specific sites below the dermal level of the skin. In particular, the transdermal delivery system is a concentrated polymer matrix that can be applied by a single sided adhesive bandage, a gauze wrap, a stretchable woven wrap, a stretchable sleeve, or a dermal adhesive dressing.

### DESCRIPTION OF THE PRIOR ART

Over the years, methods have been developed to achieve the efficient delivery of a therapeutic drug to a mammalian body part requiring pharmaceutical treatment. Use of an aqueous liquid which can be applied at room temperature as a liquid but which forms a semi-solid gel when warmed to body temperature has been utilized as a vehicle for some drug delivery since such a system combines ease of application with greater retention at the site requiring treatment than would be the case if the aqueous composition were not converted to a gel as it is warmed to mammalian body temperature. In the U.S. Patent No. 4,188,373, PLURONIC® polyols are used in aqueous compositions to provide thermally gelling aqueous systems. Adjusting the concentration of the polymer provides the desired sol-gel transition temperature, that is, the lower the concentration of polymer, the higher the sol-gel transition temperature, after crossing a critical concentration minimum, below which a gel will not form.

In U.S. Pat. Nos. 4,474,751 and 4,478,822 drug delivery systems are described which utilize thermosetting gels; the unique feature of these systems is that both the gel transition temperature and/or the rigidity of the gel can be modified by adjusting the pH and/or the ionic strength, as well as by the concentration of the polymer.

Other patents disclosing pharmaceutical compositions which rely upon aqueous gel composition as a vehicle for the application of the drug are U.S. Pat. Nos. 4,883,660; 4,767,619; 4,511,563; 4,861,760; and 5,318,780. Thermosetting gel systems are also disclosed for application to injured mammalian tissue of the thoracic or peritoneal cavities in U.S. Pat. No. 4,911,926.

Ionic polysaccharides have been used in the application of drugs by controlled release. Such ionic polysaccharides as chitosan or sodium alginate are disclosed as useful in providing spherical agglomerates of water-insoluble drugs in the Journal of Pharmaceutical Science, Volume 78, Number 11, November 1989, Bodmeier et al. Calcium alginate gel formulations have also found use as a matrix material for the controlled release of herbicides, as disclosed in the Journal of Controlled Release,(1986), pages 229-233, Pfister et al.

In U.S. Pat. No. 3,640,741, a molded plastic mass composed of the reaction product of a hydrophilic colloid and a cross-linking agent such as a liquid polyol, also containing as organic liquid medium such as glycerin, is disclosed as useful in the controlled release of medication or other additives. The hydrophilic colloid can be carboxymethyl cellulose gum or a natural alginate gum which is cross-linked with a polyol. The cross-linking reaction is accelerated in the presence of aluminum and calcium salts.

In U.S. Pat. No. 4,895,724, compositions are disclosed for the controlled release of pharmacological macromolecular compounds contained in a matrix of chitosan. Chitosan can be cross-linked utilizing aldehydes, epichlorohydrin and benzoquinone.

In U.S. Pat. No. 4,795,642, there are disclosed gelatin-encapsulated, controlled-release compositions for release of pharmaceuticals compositions, wherein the gelatin encloses a solid matrix formed by the cation-assisted gellation of a liquid filling composition incorporating a vegetable gum together with a pharmaceutically-active compound. The vegetable gums are disclosed as polysaccharide gums such as alginates which can be gelled utilizing a cationic gelling agent such as an alkaline earth metal cation.

While the prior art is silent with respect to aqueous drug delivery vehicles and isotonicity thereof, osmotic drug delivery systems are disclosed in U.S. Pat. No. 4,439,196 which utilize a multi-chamber compartment for holding osmotic agents, adjuvants, enzymes, drugs, pro-drugs, pesticides, and the like. These materials are enclosed by semipermeable membranes so as to allow the fluids within the chambers to diffuse into the environmental into which the osmotic drug delivery system is in contact. The drug delivery can be sized for oral ingestion, implantation, rectal vaginal, or ocular insertion for delivery of a drug or other beneficial substance. Since this drug delivery device on the permeability of the semipermeable membranes to control the rate of delivery of the drug, the drugs or other pharmaceutical preparations by definition, are not isotonic with mammalian blood.

To date prescriptions pain and anti-inflammatory medications which have been formulated for topical use have not been approved for sale in the United States. This is due in part to their lack of efficacy and a formulation failure to demonstrate measurable amounts of drug in the blood and urine of patients treated with these preparations. Thus proof of their ability to be transdermally transported through the skin has been heretofore unsuccessful.

In contrast, over-the-counter drugs which include counter-irritants such as menthol, eucalyptus, and camphor are solid for mild relief of minor problems. These products are designed to counter-irritation and are not intend for deep penetration of tissue structures below the skin, namely into areas which include joints, ligaments, tendons and cartilage. The over-the counter drugs described above may purchased without prescription.

A need thus exists for the administration of active therapeutic agents that can be applied topically and transported through the skin.

### SUMMARY OF THE INVENTION

The present invention relates to the formation of a stable sterile concentrated gelled composition and its use in treating acute or chronic conditions. More particularly, this invention relates to a stable, sterilized, concentrated polymer matrix, comprising a negatively charged polymer material which may be selected from the group consisting of polysulfated glucosoglycans, glycosaminoglycans, mucopolysaccharides and mixtures thereof, and a nonionic polymer which may be selected from the group consisting of carboxymethylcellulose sodium, hydroxyethyl cellulose, hydroxypropyl cellulose, and mixtures thereof.

Another embodiment of this invention involves a method for treatment of a condition in animals, which comprises topically applying therapeutically effective doses of a gelled suspension of a composition comprising an optional drug within a concentrated polymer matrix which is suspended in a liquid medium. Preferably, one of the polymer materials has a mean average molecular weight below about 800,000 and the other polymer is a nonionic cellulose derivative. The present invention utilizes a novel combination of polymers each having a specific ionicity. More specifically, the polymers used in the formulation are of two basic types: those which have a strong negative charge, and those which are non-ionic or have charge attached to them.

An alternative embodiment of the invention involves a process for the use of a composition as a medical device for drug delivery, the application of a diagnostic agent, or the prevention of post operative adhesions. This process involves topically administering to a mammal an aqueous gelled composition containing a concentrated polymer matrix composed of negatively charged polymers blended with nonionic polymers blended with nonionic polymers.

An additional embodiment involves the preparation of an antiarthritic gelled composition which comprises an NSAID drug dispersed within a matrix containing a negative charged polymer having a mean average molecular weight between about 650,000 and 800,000 blended with a nonionic polymer, wherein the molar ratio of the charged polymer is 1:0.5 to 4 and the negative charged polymer is presented in amounts of about 0.1% to about 5.0% by weight.

### BRIEF DESCRIPTION OF THE DRAWINGS

The invention may be better understood by reference to the drawings wherein:

FIG. 1 is perspective view of a dressing, with portions cut away, of one embodiment of the present invention.

FIG. 2 is perspective view of a dressing, with portions cut away, of another embodiment of the present invention (support substrate layer included).

FIG. 3 is an isometric view of FIG. 2, with release sheets in place.

FIG. 4 is a sectional view of FIG. 2, with release sheets in place.

FIG. 5 is a fragmentary view, greatly enhanced and partially in section, of a portion of the dressing as seen in FIG. 2.

FIG. 6 is a perspective view of a dressing, with a portion cut away, of an embodiment of the present invention.

FIG. 7 s a sectional view of FIG. 6.

FIG. 8 is a schematic showing the manufacture of the dressing shown in FIG. 2.

### DETAILED DESCRIPTION OF THE INVENTION

It has been unexpectedly discovered that a therapeutically effective amount of a drug may be administered topically and transdermally delivered through the skin into various sites. In order for this to be accomplished, it has been discovered that the active drug must be suspended or entrapped in a specially designed polymer matrix containing a specific molar ratio of negatively charged polymers and a non-ionic polymer suspended or dissolved in water and solubilizers.

This system is believed to form a matrix which microencapsulates, suspends and/or entraps the active drug entity such that when it is administered, it is slowly released into the systemic circulatory systems or muscular tissue providing a method of delivering an active to an affected site in the body through the skin.

The molar ratio of the polymers present in the matrix is critical in this invention. It has been found that molar ratios of the negatively charged polymer to the non-ionic polymer must be from 1:0.5 to 4, and preferably from 1:0.5 to 2.0, and most preferably from 1:0.7 to 2.5. For transdermal delivery of drugs, it has been found that ratios either higher or lower than these levels will result in a polymer shearing effect which produces unacceptable turbulence and air pockets in the composition with resulting loss of potency and efficacy. Furthermore, the solutions tend to separate and form distinct polymer layers when ionic molarity is not appropriate.

At least one of the polymers used to form the matrix of this invention must be sufficiently negatively charged to aid in the dispersion, encapsulation or solubilization of the drug. Particularly preferred polymers which have mean average molecular weights below about 800,000 and preferably molecular weights between 650,000 to 800,000 have been found acceptable to form usable polymer matrixes for transdermal delivery. Polymer with mean average molecular weights between 700,000 and 775,000 are most preferred. Polymers having molecular weights have about 800,000 form solid gels in solution and are unable to serve as part of a transdermal delivery system. Furthermore, the polymers must be sterilizable and be stable during sterilization so that the polymer does not lose molecular weight once formulated into the final transdermal delivery form.

Exemplary, non-limiting examples of compounds that may be used as a source of this molecular weight polymer include polysulfated glucosoglycans, glucosaminoglycans, and mucopolysaccharides, derivatives thereof and mixture thereof. Particularly preferred mucopolysaccharides are chondroitin sulfate and hyaluronic acid salts. Exemplary hyaluronate salts include sodium, calcium, potassium and magnesium salts with hyaluronate sodium being most preferred.

The present invention also relates to a dermal adhesive dressing as well as methods for manufacturing the dressing and using the dressing to heal and treat an animal. The dressing of the present invention is impregnated with a polymer matrix. The polymer matrix contains a supersaturated solution of hyaluronic acid alone, or in combination with other drugs, which can be topically administered to a patient in need thereof. The dressing of the present invention is able to occlusively cover the targeted area of the animal skin for treatment. This allows for better diffusion of the medication into the animal's skin.

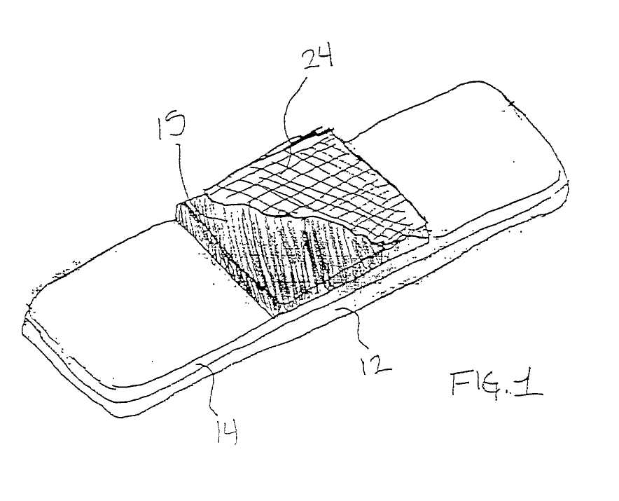

The dermal adhesive dressing of the present invention, as seen in FIG. 1, comprises a backing sheet, a polymer matrix overlying the backing sheet and a webbed covering layer overlying the polymer matrix. The upper surface of the backing sheet is coated with an adhesive which secures the polymer matrix to the backing sheet and the backing sheet to the animal skin. The polymer matrix contains a supersaturated solution of hyaluronic acid and its salts.

Another embodiment of the present invention is a dermal adhesive dressing, which comprises a backing sheet, a support substrate, a polymer matrix containing a drug or combinations of drugs and a covering layer. The upper surface of the backing sheet is coated with an adhesive, which secures the support substrate to the backing sheet. The polymer matrix is applied to the upper surface of the Support substrate. The covering layer is placed directly on top of the polymer matrix. The drug comprises a supersaturated solution of hyaluronic acid alone, or in combination with another drug or drugs. The support substrate is placed in the center of the backing sheet, and the remaining exposed area of the adhesive will be used to adhere the dressing to the animal skin. The support substrate is added to provide a desirable cushioning effect when the dressing is applied to a wound site.

In an alternative embodiment of the present invention, the dressing does not have any covering layer, and the polymer matrix is placed in direct contact with the skin of the animal. If the dressing has release sheets, the release sheets will cover the polymer matrix layer until the release sheets are removed and the polymer matrix placed against the skin.

The dressing of the present invention may also comprise one or more release sheets. Preferably, the dressing will have two release sheets. The release sheets completely cover the exposed adhesive surface of the backing sheet as well as the covering layer. The release sheets are tabbed so that they may be pulled off of the dressing prior to the application of the dressing to the skin.

The backing sheet is preferably a layer of material impervious to both oil and water, such as acetate, plastic, silicone, or the like. Ideally, the backing sheet is from about 1 ml to about 10 ml thick. The backing sheet may be formed from an inert fluorine-containing addition polymer or from poly(tetrafluoroethylene). The backing sheet may be permeable or impermeable to oil and water.

The backing sheet may be waterproof. A waterproof dressing would be desirable because it could create a seal around the wound, protecting the wound from water, germs or other environmental hazards. The backing sheet may be any color, and may also have designs or characters on it, making the dressing more acceptable to children.

The adhesive bonds the backing sheet to the support substrate as well as the skin of the animal. The adhesive is pressure sensitive. Preferably, the adhesive is a medical grade silicone adhesive which will not be solubilized by the polymer matrix.

If the dressing includes a support substrate, the support substrate may be a woven fabric. The support substrate may also be a non-woven fabric, such as polyester, nylon or a polyester nylon blend. The support substrate may also be a knitted fabric or a foam.

The support substrate may be cut to a size which covers only the area of the skin which is being treated. In another embodiment of the present invention, additional layers of support substrate may be added under the polymer-matrix in order to give the dressing a more quilted, comfortable feel.

In the preferred embodiment of the present invention, a covering layer is used to reinforce the polymer matrix for application to the skin of the patient. The fabric of the covering layer should be elastic, or a fibrous or porous sheet material such as cotton or polyester felt, or the like which will allow for good bonding during the impregnation process and also is somewhat elastic in nature. Preferably, the fabric which is used as the covering layer of the present invention is non-woven and porous. More preferably, the covering layer may be a polymer selected from the group consisting of polyvinyl chloride, polyethylene, polypropylene, polyester and nylon. Preferably, the covering layer has a percent open area of least 20% but no greater than 88%. When the porous covering layer is placed over the polymer matrix, the drug is pushed into the pores and dispersed throughout the covering layer. The circumference of the covering layer is greater then the circumference of the polymer matrix to allow for the increased diameter of the polymer matrix when pressure or shearing forces are applied to the dressing.

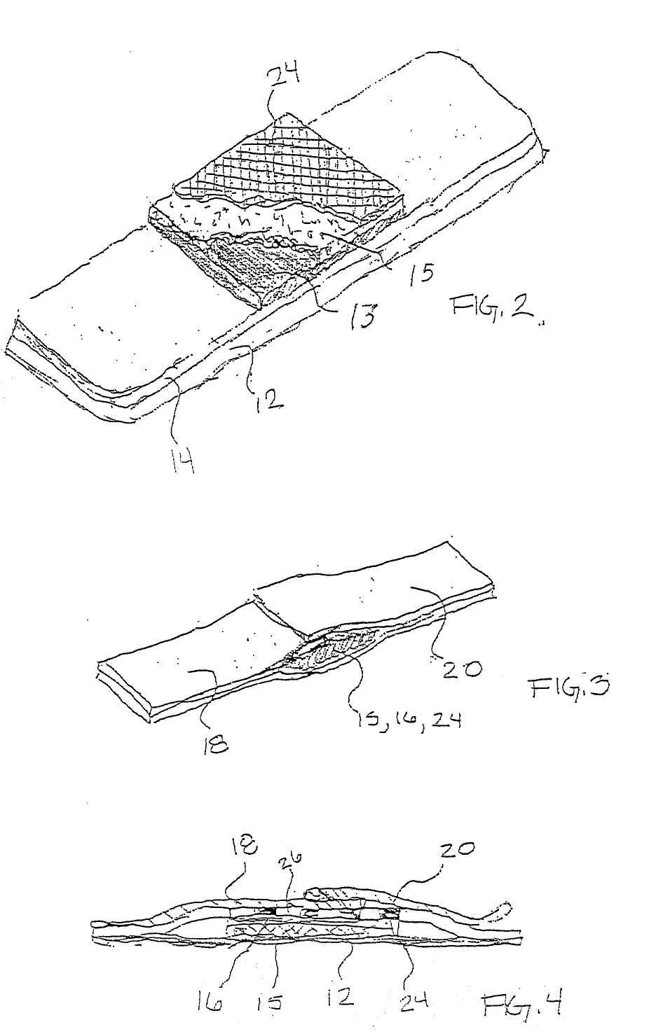

The release sheets may be formed from an inert fluorine-containing addition polymer. The release sheet or sheets should extend beyond the edge of the covering layer, as seen in FIGS. 2 and 3, to provide grasping tabs with which to remove them from the dressing before use.

The present invention further comprises a dressing impregnated with an ionic polymer matrix containing a supersaturated solution of hyaluronic acid and its salts. In particular this polymer matrix is useful for wound healing, treating acute or chronic intractable pain, cancer therapy and treating diabetic ulcers in the animal. The invention involves the use of specialized compounds manufactured by using polymers of average molecular weights below about 800,000 in a unique process for the creation of specially modified molecules to treat a variety of conditions. The polymer matrix used in the present dressing is suspended or solubilized in water with various drugs. The polymers must be sterilizable and acceptable for animal and human use.

The polymer matrix can be contoured during manufacture resulting in a matrix of variable thickness and curvature. Similarly, the polymer matrix can be contoured to form a matrix of variable thickness with a central area of zero thickness where an aperture can be created. The matrix can also be of uniform thickness. The thickness of the polymer matrix can be from 0.01 to 1.0 cm, or thicker if desired. The ionic polymer matrix is highly flexible, and can conform to the shape of the skin and surrounding area being treated so as to apply a drug in a prescribed and even manner

Hyaluronic acid (HA) occurs naturally in joint synovial fluid, where it plays a lubricating role, and may have biological activity was well. HA is a mucopolysaccharide, and may alternatively be referred to as glycosaminoglycan. The repeating unit of the hyaluronic acid molecule is a disaccharide consisting of D-glucuronic acid and N-acetyl-D-glucosamine. Because hyaluronic acid possesses a negative charge at neutral pH, it is soluble in water, where it forms highly viscous solutions. The D-glucuronic acid unit and N-acetyl-D-glucosamine unit are bonded through a glycosidic, beta (1-3) linkage, while each disaccharide unit is bonded to the next disaccharide unit through a beta (1-5) linkage. The beta (1-4) linkages may be broken through hydrolysis with the enzyme hyaluronidase.

A variety of substances, commonly referred to as hyaluronic acid, have been isolated by numerous methods from various tissue sources including umbilical cords, skin, vitreous humour, synovial fluid, tumors, haemolytic streptocci pigskin, rooster combs, and the walls of veins and arteries. It has also been synthesized artificially and by recombinant technology.

Conventional methods for obtaining hyaluronic acid results with a product having differing properties and a wide range of viscosities. U.S. Pat. No. 2,585,546 to Hadian, discloses an example of a method for obtaining hyaluronic acid which involves extracting acetone-washed umbilical cords with a dilute salt solution, acidifying the resulting extract, removing the colt so formed, precipitating some hyaluronic acid with protein from the acidified extract with ammonium sulfate, agitating the liquid with pyridine, precipitating another fraction highly contaminated with protein, followed by more ammonium sulfate which forces some pyridine out of solution along with the high viscosity hyaluronic acid. The hyaluronic acid collects at the interface between the two liquid phases and may be separated by filtration, centrifugation or another usual procedure. A modification of this process involves the fractionation of the acidic salt extract from the umbilical cords with alcohol and ammonium sulfate. Alcohol is added to the acidic salt extract, and resulting precipitate is removed. Solid ammonium sulfate is added to the liquid until saturation and the solution forms two phases with a precipitate of hyaluronic acid at the interface.

U.S. Pat. No. 4,517,296 to Brace et al. is directed to the preparation of hyaluronic acid in high yield from Streptococcus bacteria under anaerobic conditions in a CO² enriched growth medium, separating the bacteria from the resulting broth and isolating hyaluronic acid from the remaining constituents of the broth. Separation of the microorganisms from the hyaluronic acid is facilitated by killing the bacteria with trichloroacetic acid. After removal of the bacteria cells and concentration of the higher molecular weight fermentation products, the hyaluronic acid is isolated and purifies by precipitation, resuspension and reprecipitation.

One particular fraction of hyaluronic acid (HA) that exhibits excellent matrix formation according to the present invention is hyaluronic sodium having a mean or average molecular weight between 650,000-800,000, preferably 700,000-775,000 with a high decree of purity, 95-105% free, and preferably at least 98% pure, from contamination of related mucopolysaccharides. Furthermore, this hyaluronic acid has a sulphated ash content of less than 15% and a protein content of less than 5%. Examples of usable base salts include those safe for animal and human use, such as sodium, potassium, calcium, and magnesium salts or the like.

In contrast to HA, chondroitins are mucopolysaccharides comprising repeating units of D-glucuronics acid and N-acetyl-D-galactosamine. Chondroitin sulphates are important components of cartilage and bone and are excellent for preparing the polymer matrix herein.

The negative charged polymers are generally present in the system in amounts which enables a solid gel to be formed. Generally, gels are formed using amounts of about 2.0 to about 3.0% by weight with amounts of about 2.1 to about 2.5% by weight being preferred for use as a topical gel.

The solutions used to prepare the gel of the present invention may be prepared in a variety of ways. For example, the polymers may be dissolved in water and purified either separately or jointly and then the optional active drug added to the system.

A particularly preferred procedure involves separately dissolving the nonionic polymer in water and centrifuging the material to form a solution and then remove impurities. This may be conveniently done at rotation speeds of 2000 rpm for times of about 30 minutes to about two hours.

In contrast, the negative charged polymer may be blended and stirred in water until it is dissolved. This process must be done while avoiding the formation of bubbles and while freeing the polymer of its electrostatic activity. Furthermore, the molecular weight of the polymer must not be significantly changed during processing and as such mild process conditions are required. Processing conditions of 400-3000 rpm for durations of 16-24 hours have been found acceptable to produce stable solutions or gels of the charged polymer.

Conventional pharmaceutically acceptable emulsifiers, suspending agents, antioxidant (such as sodium metabisulfate) and preservatives (such as benzyl alcohol)may then be added to this system. Once all the components are blended together, such as by mixing 400-3000 rpm for one to four hours, the system is filled into tubes and sterilized. The resulting system is filled into tubes and sterilized. The resulting system is a clear gel which is storage stable for several years.

The drug may be added to the homogenous solution or gel separately once dissolved or disbursed in water. Emulsifiers, suspending agents and preservatives may be then added to this system. One particularly nonlimiting effective material for solubilizing water insoluble drugs is methoxypolyethlene glycol (MPEG). Once all the components are blended together, for 400-3000rpm for 1 to 4 hours, the system is filled into tubes and sterilized. The resulting system is storage stable for several years.

The formulations may be used topically and also contain conventional pharmaceutical acceptable excipients well known to those skilled in the art, such as surfactants, suspending agents, emulsifiers osmotic enhancers, extenders and dilutants, pH modifiers as well as fragrances, colors, flavors and other additives.

As indicated above, the active drug agents may be blended with the aqueous polymer matrix at the time of manufacture. As such, the drug when in the form of a water-soluble solid is simply diluted with sterilized water or polymer matrix solution and prepared in gel form.

The dosage system can be formed with or without the use of pharmaceutically acceptable preservatives. A significant advantage of the dosage form of the present system relates to its ability to allow the drug to slowly diffuse through tissue when administered thus allowing for an effective for an effective therapeutic dose to be present for many house.

In this regard, it should be noted that reference to therapeutically effective doses does not necessarily relate to conventional dosage levels, but does relate to drug levels that achieve an effective therapeutic level at the dose employed, which may be the same level but not at the same frequency of administration previously required for drugs taken orally or by injection. This not only significantly reduces the number of doses required to achieve the same effect, but it also reduces costs, maintenance and health hazards associated with conventional treatment therapies. Additionally, it results in immediate and continued drug release for long periods of time spanning several hours in-duration.

Doses may vary from patient to patient depending on the type and severity of the condition being treated and the drug being administered. Generally, doses of 1 ml to 75 ml may be administered with preferred doses using 2 to 25 ml of the gelled matrix system.

### Pain Applications

The formulations of this invention may be used to treat a variety of mammal and animal conditions and physical state. One system having a particular application relates to pain management, namely the prevention, treatment and alleviation of pain associated with any disease condition or physical state.

Without being limited to the specific pain treated, the preparations of this invention may treat the following nonlimiting locations or sources of pain below the dermal level of the skin, including, but not limited to knees, ankles, hands, feet and neck.

The importance of this invention becomes apparent when one considers the side-effects associated with conventional, oral drugs for treating osteoarthritis, including NSAIDs such as diclofenac.

Typically, NSAIDs have been known to produce gastric and intestinal irritation. In addition, scarring and ulceration of intestinal tract is quite common inpatients on short- or long-term NSAID therapy. Unfortunately, there do not appear to be many alternatives to NSAID therapy, for patients suffering from extremely painful, inflammatory conditions which may include osteoarthritis and other inflammatory disorders. Thus, new NSAIDs are constantly entering the market place, each one, however, with the same potential to cause unpleasant and often serious side-effects.

The transdermal applications of NSAIDs and particularly diclofenac described herein, are a much safer way of treating inflammatory disorders including those related to osteoarthritis also known as Degenerative Joint Disease (DJD).

When a person takes an oral form of diclofenac, typically 100 mg to 150 mg per day,, the drug must be circulated through systemic blood and only a small amount ends up in the specific site that is intended for treatment, such as the knee. Individuals with osteoarthritis are generally treated with NSAIDs including, but not limited to, diclofenac, ibuprofen, Aspirin, etc., which as previously mentioned produce an anti-inflammatory effect at the joint level. At therapeutic dosages for diclofenac which are usually between 100 mg and 200 mg per day, more than 50% of all treated patients will experience some form of GI (gastrointestinal) distress.

The transdermal delivery system described herein offers a major alternative especially for those individuals who have a history of undesirable side-effects associated with gastric and intestinal irritation. Also for those patients who have already suffered damage, including ulceration and loss of absorption from the intestinal tract, the transdermal preparations described herein present a new way of providing effective treatment and relief of painful symptoms. It has become a common practice of rheumatologists and other specialists treating osteoarthritic and associated disorders to use ulcer-type drugs of the H2 blocking variety including, but not limited to ranitidine (Zantac), Pepsid and cimetidine (Tagamet) by Smith Kline. The addition of these drugs to already high regiments “(polypharmacy)” of therapeutic agents is not desirable since these drugs often produce their own undesirable side-effects. Although an occasional patient will experience mild stomach upset from the transdermal preparation described herein, the effect is transient and of mile severity. In addition, patients treated with the present transdermal diclofenac, find that they can function for longer periods of time (4 to 6 hours) and can simply apply more of the therapeutic gel to maintain a continuous reduction in-pain and inflammation. In this way, patients who apply the drug topically 3 to 4 times a day can experience sustained around-the-clock relief.

Several attempts have been made in the past to produce effective transdermal preparations. These preparations have not been approved in North America for some drugs, like diclofenac, by the regulatory authorities as of this time. Some of the reasons cited are lack of proven transdermal delivery. In the case of the current invention, transdermal delivery can be substantiated by:

1. Measurable blood levels of diclofenac.

2. Diclofenac presence in the urine of patients treated with the transdermal drug.

3. The pressure of diclofenac in synovial fluid where joints with synovial fluid are the target sites for treatment.

4. Rapid absorption following topical administration.

5. Rapid relief of painful symptoms in a significant number of patients already being treated with the products.

In Europe, Voltaren cream (Ciba-Geigy) is popular for the treatment of osteoarthritic conditions. This preparation contains diclofenac sodium. However, the manufacturers have not demonstrated to the satisfaction of North American regulators proven ability for the cream to be transdermally absorbed. Amounts of diclofenac delivered by the cream are considered to be minimal at best.

It should be pointed out that diclofenac, as the sodium or potassium salt, is a benzeneacetic acid derivative, designated chemically as 2-[2,6-di-chlorophenyl)amino]benzeneacetic acid, monosodium or monopotassium salt. It is freely soluble in methanol, soluble in ethanol, and practically insoluble in chloroform and in dilute acid. Diclofenac sodium is sparingly soluble in water while diclofenac potassium is soluble in water. Diclofenac, the anion in Voltaren® and Calaflam®, is a nonsteroidal anti-inflammatory drug (NSAID). In pharmacologic studies, diclofenac has shown anti-inflammatory, analgesic, and antipyretic activity. As with other NSAIDs, its mode of action is not know; its ability to inhibit prostaglandin synthesis, however, may be involved in its anti-inflammatory activity, as well as contribute to its efficacy in relieving pain related to inflammation and primary dysmenorrhea. With regard to its analgesic effect, diclofenac is not a narcotic.

The current invention represents a break-through in that for the first time measurable, detectable levels of diclofenac can be delivered to affected sites. For those patients who experience mild intestinal discomfort following administration, it is recommended that the transdermal gel preparation described herein, be administered after meals.

In addition to the negatively charged polymers, the transdermal polymer matrix must contain a non-ionic polymer which facilitates in retarding the absorption of the active drug through the skin and delays or slows down in animals natural absorption of the negatively charged polymer.

Without the presence of this component, the active drug would not be delivered transdermally into the site targeted for treatment at levels which are therapeutically effective. In addition to the non-ionic polymers described in this system, these materials are necessary to provide thorough penetration of skin layers including the epidermis, dermis and fatty tissue layers. Evidence of this absorption through the skin layers and into the capillary bed and ultimately the systemic system is evidenced by the fact that detectable, measurable blood levels of active drug, such as diclofenac, can be found in the urine of patients treated with the diclofenac transdermal preparation described herein.

Test Procedure I

Patient LHN's complaint is of headache and pain in the back of the neck.

History

She has been getting headaches for 30 years since she was 5-years-old. She has several injuries in the past including being thrown down the stairs.

In 1996, it was noted that the headaches were bifrontal, sometimes behind the eyes and also in the sides of the head and in the parietal region. They were often associated with nausea and vomiting.

In June 1996, her headache was frontal, occipital and in the left shoulder going down the left arm, and she also had low back ache.

Physical Examination

She was tender over the right cervical facets at 2-3, 4-5 and 5-6 and on the left at 2-3 and the greater occipital nerve bilaterally.

Diagnosis

Cervicogenic headaches.

This was confirmed by diagnostic blocks bilaterally at 2-3, 3-4 and 4-5 which reduced her head and neck pain respectively of 6/10 and 10/10 to 0/10.

Treatment With Diclofenac Gel

This was rubbed on the facet joint areas of the cervical spine bilaterally. The patient noticed marked decrease of pain in the neck 4 to 8 hours after use.

When the gel was used 2 to 3 times daily, the generalized neck ache was markedly reduced. In addition, some of her headaches were also decreased. It was noted that there was not skin irritation with the use of the gel.

Test Procedure II

This is a 32-year-old man who complains of headaches.

History

He complains of headaches in the right upper neck radiating to the right parietal region, the right eye, the right temporal region. They are aching and stabbing with a severity between 6-10/10. They are always present but the severity varies. They have occurred since he had a motor vehicle accident in August 1993.

Physical Examination

Flexion normal, extension 80%, rotation right 90% and rotation left 90%. He is tender at the cervical facets of right 2-3, left 2-3 and the right lesser occipital nerve.

Diagnosis

Cervicogenic headache.

This was confirmed by a positive response to diagnostic facet blocks at the right 2-3 and 3-4 cervical facets.

Treatment With Diclofenac

This was rubbed on the facet joint area on the right side of the neck and the patient noticed a marked decreased in pain for the next 4 to 8 hours after use. When the gel was used 2 or 3 times a day, the generalized neck ache was markedly reduced. In addition, some of his headaches were also decreased.

Test Procedure III

Her complaint is of severe holo-cranial headaches.

History

She gave a history that one and one-half years ago she feel flat on her back on concrete. She has had severe headaches since then although earlier in her life she had headaches that were attributed to migraine.

She is 37-years-old. The headaches are biparietal, temporal, behind the eyes and alter in the day they become bioccipital. They have an aching and throbbing character. Sometimes she wakes up with a headaches.

She has had some success with Fiorinal C½ in treating her headaches.

Physical Examination

Neck: Flexion 305, extension 40%, right rotation 80% and left rotation 70%. Tenderness of the cervical facets, right 2-3 and 3-4 and left 2-3, 3-4, 4-5 and 5-6, 1+ at each.

Diagnosis

Cervicogenic headache.

Possible pre-existing migraine.

Treatment With Diclofenac Gel

This was rubbed on the facet joint areas bilaterally in the neck region. The patient noticed a marked decrease in pain in the neck for 4 to 8 hours after use. When the gel was used 2 to 3 times a day, the generalized neck pain was markedly reduced. In addition, some of her headaches were also decreased.

Test Procedure IV

This 52-year-old lady had a long history of:

Occasional headaches.

Occasional neck pain.

History

The patient had a long history of headaches of about 30 years duration. These were of a migrainous nature usually on the right side. More recently, these have been associated with neck pain.

Physical Examination

This revealed a tilt of the head to the left. With the right should higher than the left.

The facet joints at C2-3, C3-4, C4-5 and C5-6 bilaterally were very tender. However, they were particularly tender at C2-3 and C4-5 on the right.

Diagnosis

Degenerative joint disease of the cervical spine causing chronic headaches and occasional neck aches.

Results of Treatment With Diclofenac Gel

This was used on three occasions for the neck pain. In each case, it decreased the neck pain substantially. On two occasions, it aborted a migraine headache in its early stages.

Test Procedure V

This 47-year-old lady has a long history of:

1. Constant headaches.

2. Constant neck aches.

History

The patient has a history of 7 motor vehicle accidents. She underwent facet rhizolysis about three years ago. This almost entirely relieved her headaches. She still however continued to have neck aches with physical activity particularly involving the neck.

Physical Examination

This showed some limitation of flexion and extension to about 65% of normal. The facet joints from C2 to C6 were exquisitely tender more on the right than the left.

Diagnosis

Degenerative joint disease of the cervical spine causing occasional headaches and neck aches.

Treatment With Diclofenac Gel

The diclofenac gel has successfully relieved her neck ache on three different occasions. Each time the pain relief was almost 100%. In addition, it stopped the beginnings of a headache on each occasion.

Test Procedure VI

This 26-year-old lady has a long history of:

1. Constant neck ache.

2. Almost daily headaches.

History

The patient was thrown off a friend's shoulders while playing at a party. She landed on her jaw and had her neck thrust backwards violently.

She was thought to have actually broken her jaw at the time of the fall.

She has been investigated for TMJ disorder because there is clearly some asymmetry in her face since the accident. However, the TMJ specialist felt that there was no TMJ damage that could be found.

She also was found to have tender facet joints from C2 to C6 bilaterally, and she said with her neck thrust forward and with difficulty in flexion and extension particularly extension being only about 60% of normal.

Physical Examination

This revealed tenderness over the facet joints at C2-3, C3-4, C4-5 and C5-6 bilaterally but especially on the right. And the facet joints were more prominent on the right.

The TMJ was not especially tender to palpation.

Diagnosis

Degenerative joint disease of the cervical spine causing chronic neck aches and headaches.

Treatment With Diclofenac Gel

This was used on three occasions for severe neck pain. It decreased the neck pain by about 50%. It did not however relieve the headaches. The patient is now using the gel daily because she does find that it cuts down her neck pain, and she is hoping it will cut down the headaches.

The invention being thus described, it will be obvious that the same may be varied in many ways. Such variations are not to be regarded as a departure from the spirit and scope of the invention and all such modifications are intended to be included within the scope of the following claims.

### Dermatological Applications

In addition to treating disorders associated with pain below the dermal level of the skin, the preparations of this invention may be used to treat a wide variety of dermatologic disorders. Exemplary, non-limiting disorders include dermatitis conditions such as: Contact Dermatitis: Atopic Dermatitis; Seborrheic Dermatitis; Nummular Dermatitis; Chronic Dermatitis of Hands and Feet; Generalized Exfoliative Dermatitis; Stasis Dermatitis; and Localized Scratch Dermatitis; bacterial infections of the skin, such as: Staphylococcal Diseases of the skin, Staphylococcal Scalded Skin syndrome; Erysipelas; Folliculitis; Furuncles; Carbuncles; Hidradenitis Suppurativa; Paronychial Infections and Erythrasma; superficial fungal infections such as: Dermatophyte Infections; Yeast Infections; Candidiasis; and Tinea Versicolor; parasitic infections of the skin such as Scabies; Pediculosis; and Creeping Eruption; disorders of hair follicles and sebaceous glands such as: Acne; Rosacea; Perioral Dermatitis; Hypertrichosis; Alopecia; Pseudofolliculitis Barbae; and Keratinous Cyst; scaling papular diseases, such as: Psoriasis; Pityriasis Rosea; and Lichen Planus; pressure sores; benign tumors and malignant tumors.

### Wound Applications

Additional disorders to be treated are pressure sores. Factors that precipitate pressure sores include loss of pain and pressure sensations (which ordinarily prompt the patient to shift position and relieve the pressure) and the thinness of fat and muscle padding between bony weight-bearing prominences and the skin. Disuse atrophy, malnutrition, anemia, and infection play contributory rate. Spasticity, especially in patients with spinal cord injuries, can place a shearing force on the blood vessels to further compromise circulation.

The most important of the extrinsic factors is pressure. Its force and duration directly determines the extent of the ulcer. Pressure severe enough to impair local circulation can occur within hours in an immobilized patient, causing local tissue anoxia that progresses, if unrelieved, to necrosis of the skin and subcutaneous tissues. The pressure is due to infrequent shifting of the patient's position; friction and irrigation from ill-adjusted supports or wrinkled bedding or clothing may be contributory. Moisture, which may result from perspiration or from urinary or fecal incontinence, leads to tissue maceration and predisposes to pressure sores.

The stages of decubitus ulcer formation correspond to tissue layers. Stage 1 consists of skin redness that blanches or disappears on pressure; the skin and underlying tissues are still soft. Stage 2 shows redness, edema, and induration, at times with epidermal blistering or desquamation. In stage 3, the skin becomes necrotic with exposure of fat and drainage from wound. In stage 4, necrosis extends through the skin and fat to muscle; further fat and muscle necrosis characterizes stage 5. In stage 6, bone destruction begins, with periostitis and osteitis, progressing finally to ostemyelitis, with the possibility of septic arthritis, pathologic fraction and septicemia.

The best known treatment for pressure sores is prevention. Pressure on sensitive areas must be relieved. Unless a full flotation bed (water bed) is used to provide even distribution of the patient's weight through hydrostatic buoyancy, the bedridden patient's position must be changed at least once every 2 hours until tolerance for longer periods can be demonstrated (by the absence of redness). Air-filled alternating-pressure mattresses, sponge-rubber “egg-crate” mattresses, and silicone gel or water mattresses decrease pressure on sensitive areas but do not negate the need for position changes. A turning (Stryker) frame facilities turning patients with cord injuries. Protective padding (e.g., sheepskin or a synthetic equivalent) at bony prominences should be used under braces or plaster casts, and at potential pressure sites a window should be cut out of the cast. A wheelchair patient must be able to shift his position every 10 to 15 minutes even if he is using a pressure-relieving pillow. Otherwise, patients in chairs may be more likely to have pressure sores than those who are in bed.

The major problem in treating decubitus ulcer is that the ulcer is like an iceberg, a small visible surface with an extensive unknown base, and to date there is no good method to determine the extent of tissue damage. Ulcers that have not advanced beyond stage 3 may heal spontaneously if the pressure is removed and the area is small.

Stage 4 ulcers require debridement; some may also require deeper surgery. When the ulcers are filled with pus and necrotic debris, application of dextranomer beads or other and newer hydrophilic polymers may hasten debridement without surgery. Conservative debridement of necrotic tissue with forceps and scissors should be instituted. Some debridement may be done by cleansing the wound with 1.5% hydrogen peroxide. Wet dressings of water (especially whirlpool baths) will assist in debriding. The granulation that follows removal of necrotic tissue may be satisfactory for skin grafts to cover small areas.

More advanced ulcers with fat and muscle involvement require surgical debridement and closure. Affected bone tissue requires surgical removal; disarticulation of a joint may be needed. A sliding full-thickness skin flap graft is the closure of choice, especially over large bony prominences (e.g., the trochanters, ischia, and sacrum), since scar tissue cannot develop the tolerance to pressure that is needed.

For spreading cellulitis, a penicillinase-resistant penicillin or a cephalosporin is necessary.

Peripheral vascular distress due to diabetes is also treatable. The primary cause of ulceration in diabetic patients is the occlusion of the blood supply to the extremities, as well as sensory denervation. Both factors contribute to the impaired ability of the patient to perceive trauma which has occurred, thus possibly causing a compounding of the damage due to lack of timely treatment. However, even with prompt treatment, reduced blood supply combined with decreased cellular immunity greatly increase the risk of fungal and bacterial infections.

Treatment of peripheral vascular distress due to diabetes is complex, because management of the underlying condition is primary. Further, stabilization of the diabetic condition alone will not necessarily alleviate the ulcerations. If possible, patients are advised to avoid weight bearing activities and appropriate orthotic protection applied. Production and application of a cushioned layer of the matrix incorporating the appropriate antiviral and/or-antibiotic agent is an effective as well as efficient treatment.

### Motion Sickness Applications

Difficulties experienced in adaptation to various forms of travel or movement are also treatable via embodiments of the present invention. Motion sickness is caused by excessive stimulation of the vestibular apparatus during motion. While the complete physiological mechanism is not fully understood, it is believed that a combination of visual stimuli, poor ventilation and emotional factors precipitate attacks of motion sickness.

It is generally believed that treating person susceptible to motion sickness prior to onset of symptoms produces a greater reduction in the severity of distress than treatment after symptoms have developed. Due to the complexity and combined nature of the symptoms, a variety of treatment options may be employed. Drugs such as dimenhydrate, diphenhydramine, meclinzine, cyclizine, promethazine, diazepam and scopolamine, as well as phenobarbital, in the case of psychological distress, may be employed.

Utilization of a dermal patch produced with an effective motion sickness medicament applied approximately one to four hours prior to exposure to precipitating factors can deliver an effective and prolonged dosage. If extended exposure to travel is anticipated, a dermal patch produced with a more appropriate dosage amount may be administered.

### General Applications

Many new dressings and topical agents are being tested and made available tor use. No one powder, gel, or dressing is universally superior. The subject is complex; i.e., some are wet and lead to Pseudomonas infection if used to long, others are painful, all are expensive, and some are of little value.

Use of the present formulations either alone or in combination with various therapeutic agents overcomes all of these prior are deficiencies.

It has also been unexpectedly found that when the system is administered in a repetitive manner, once the effects of the active drug are reduced in intensity or effectiveness, such repeat treatments may result in a synergistic effect by enhancing the initial term of relief to a period when exceeds the initial time of relief. This is also experienced on subsequent treatments. In this way, the present formulations are able to extend relief or treatment from normally several hours to at least several days of relief. The use of repeat applications enhances drug release which significantly reduces drug dependence. It also results in the relief of continued tissue damage and may even assist in tissue repair.

Regardless of the route of administration elected, the formulations of the present invention are formulated into pharmaceutically acceptable dosage forms by conventional methods known in the pharmaceutical art.

As discussed above, an effective but nontoxic amount of the system is employed in treatment. The dose regimen for administering drugs or treating various conditions, such as pain as described above, is selected in accordance with a variety of factors including the type, age, weight, sex, and medical condition of the subject, the severity of the pain, the route of administration and the particular complex or combination of drugs employed. Determination of the proper dose for a particular situation is within the skill of the art. Generally, treatment is initiated with smaller dosages which are less than the optimum doses of the compound. Thereafter, the dose is increased by small increments until the optimum effect under the circumstances is reached. For convenience, the total daily dosage may be divided and administered in portions during the day if desired. Generally, amounts of matrix with or without drug may vary from 0.0001% to about 75% by weight of the system when using topically with 2 to 25 ml concentrations and preferably in 3 to 10 ml amounts.

The formulations of this invention are particularly useful in the administration of drugs that could be previously administered only orally.

Particularly preferred nonionic polymers are cellulose derivatives and particularly those selected from the group consisting of carboxymethylcellulose sodium, hydroxyethyl cellulose, hydroxypropyl cellulose and mixtures thereof. These particular polymers have been found to posses exceptional ability to form sustained release matrix formulations when used in combination with a negatively charged polymer. Such polymers are generally employed in amounts of about 0.1% to about 1.0% and preferably about 0.5 to about 1.0%. Amounts above about 1.0% result in the formation of a solid gel when used with the negatively charged polymer. Amounts below about 0.1% have not been found suitable to prepare a storage stable product that has sustained drug release.

A particularly preferred HEC concentration is about 0.2% to about 1.0% by weight of the matrix.

A wide variety of medicaments which may be administered topically may be used in the delivery system according to this invention. These include drugs from all major categories, and without limitation, for example, anesthetics including benzocaine, tetracaine, mepivacaine, prilocaine, etidocaine, bupivacaine and lidocaine; analgesics, such as acetaminophen, ibuprofen, fluriprofen, ketoprofen, voltaren (U.S. Pat. No. 3,652,762), phenacetin and salicylamide; nonsteroidal anti-inflammatories (NSAIDS) selected from the group consisting of naproxen, acetaminophen, ibuprofen, flurbiprofen, ketoprofen, phenacetin, salicylamide, and indomethacin; antibiotics including amebicides, broad and medium spectrum antibiotics, fungal medications, and anti-viral agents and specifically including such as erythromycin, penicillin and cephalosporins and their derivatives; central nervous system drugs such as thioridazine, diazepam, meclizine, ergoloid mesylates, chlorpromazine, carbidopa and levodopa; metal salts such as a potassium chloride and lithium carbonate; minerals selected from the group consisting of iron, chromium, molybdenum and potassium; immunomodulators; immunosuppressives; thyroid preparations such as synthetic thyroid hormone, and thyroxine sodium; steroids and hormones including ACTH, anabolics, androgen and estrogen combinations, androgens, corticoids and analgesics, estrogens, glucocorticoid, gonadotropin, gonadotropin releasing, human growth hormone, hypocalcemic, menotropins, parathyroid, progesterone, progestogen, progestogen and estrogen combinations, somatostatis-like compounds, urofollitropin, vasopressin, and others; and vitamins selected from water-soluble vitamins such as B complex, vitamin C, vitamin B12 and folic acid and veterinary formulations.

Chemotherapeutics such as Actinomycin D, adriamycin, altretamine, asparaginase, bleomycin, busulphan, capecitabine, carboplatin, carmustine, chlorambucil, cisplatin, cyclophosphamide, cytarabine, dacarbazine, daunorubicln, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, methotrexate, mitomycin, mitozantrone, oxaliplatin, procarbazine, steroids, streptozocin, taxol, taxotere, tamozolomide, thioguanine, thiotepa, tomudex, topotecan, treosulfan, vinblastine, vincristine, vindesine, vinorelbine, and the like may be empolyed. Other chemotherapeutics useful in combination and within the scope of the present invention are buserelin, chlorotranisene, chromic phosphate, dexamethasone, estradiol, estradiol valerate, estrogens conjugated and esterified, estrone, ethinyl estradiol, floxuridine, goserelin, and prednisone.

One particular criteria of the drug is that they must be solubilized in the polymer matrix solution in order to be topically administered.

A particularly preferred additional use of the compositions of this invention include their uses as 1) a medical device, 2) for drug delivery, 3) the application of a diagnostic agent or 4) the prevention of post operative adhesions.

### Concentration Potentials

One useful aspect of the present invention is that of concentrating the matrix to various degrees depending upon intended usage. The step of concentrating, must be practiced under conditions that avoid degradation of the hyaluronic acid and its salts. These conditions can be determined without undue experimentation by a person of ordinary skill in the art. Concentrating is generally practiced until between about 10 percent by weight and about 70 percent by weight, and preferably until between about 20 percent by weight and about 50 percent by weight, of the water is removed from the polymer matrix.

A number of techniques may be employed to dehydrate the polymer matrix ranging from the use of solvents and rotary evaporation to heating either a previously prepared hyaluronic acid solution or the polymer matrix itself. Preferably, the water removal step is affected by controlling of the temperature of the solution. Widely varying temperatures can be employed for the concentrating step, however, the temperature is generally maintained from about 10° C. to about 80° C. and preferably from about 30° C. to about 60° C. subatmospheric pressure may also be used. While, superatmospheric pressure is suitable, this step is preferably practiced at atmospheric pressure, namely about 760 mmHg.

One method of concentrating the polymer matrix is by supersaturating the hyaluronic solution after blending with a non-ionic polymer. For example, the polymers may be dissolved in water and purified either separately or jointly and then an optional drug is added to the system.

Another method for preparing a concentrated hyaluronic acid solution is by slowly adding hyaluronic acid to sterilized water being stirred at approximately 200-600 rpms. The molecular weight and purity of the hyaluronic acid as described previously are of the upmost importance and must not be significantly changed during processing, therefore mild processing conditions are required. Stirring is continued until the HA has completely dissolved into the water and a crystal clear viscous solution has formed. Next, a quantity of the solution is removed and placed in a clean vessel, where constant stirring is continued. The vessel is then placed in a warm environment and is monitored. The water content is removed by evaporation without causing the molecular degradation of the HA. The amount of water removal may be determined by the weight reduction of the solution. If weighing the solution does not indicate the desired amount of water either present or removed, the vessel may be returned to the warm environment for further water removal.

Again, 10% to about 70% of the water may be removed from the solution, with a preferred range of 37%. When 37% of the water is removed, preferably, the supersaturated solution of hyaluronic acid is present in the polymer matrix in an amount from about 37% to about 40.1% by weight. More preferably, the supersaturated solution of hyaluronic acid is present in the polymer matrix in an amount from about 37.2% to about 39.2% by weight. Even more preferably, the supersaturated solution of hyaluronic acid is present in the polymer matrix in an amount from about 37.6% to about 38.9% by weight.

According to another aspect of the present invention the composition further comprises an active therapeutic agent. Any active therapeutic agent which is compatible with hyaluronic acid and its salts can be employed in the present invention. A wide variety of medicaments which are administered may be used in the delivery system according to this invention.

### Dressing Preparations

Sodium hyaluronate (NAHA) is a major carbohydrate component of the extracellular matrix and can be found in skin, joints, eyes and most other organs and tissues. It has a linear co-polymer structure that it is completely conserved throughout a large span of the evolutionary tree, indicating a fundamental biological importance. Amongst extracellular matrix molecules, it has unique hydroscopic, Theological and viscoelastic properties. Sodium hyaluronate binds to many other extracellular matrix molecules through its complex interaction with matrix components and cells.

Furthermore it is believed that sodium hyaluronate has an important biological role in skin wound healing, by virtue of the fact that during wound healing levels of sodium hyaluronate are elevated temporarily in granulation tissue. It has been determined that there is a specific binding interaction between fibrin, the major clot protein, and sodium hyaluronate which is a constituent of the wound extracellular matrix. The binding interaction may provide the driving force to organize a three-dimensional NAHA matrix which attaches to the fibrin matrix.

Sodium hyaluronate-fibrin matrix plays a major role in the subsequent tissue reconstruction processes. Traumatic wounds, such as those caused by surgical procedures, often produce irregular patterns on NAHA-fibrin matrix and the biological functioning of the system wounds is often compromised.

One purpose of providing hyaluronate acid derived matrix, which is unique to the polymer matrix, is to facilitate would healing and to prevent complications such as those found when scarring and adhesions are formed. In other words, the prime purpose of providing an impregnated web or padded web with a stable, transdermal sodium hyaluronate matrix in the form of the polymer matrix complex is important in regulating the molecules which control cellular function and which are involved in the inflammatory response and new blood vessel formulation amongst other factors which are involved in wound healing.

The body of work in the scientific literature amounting to many hundreds of studies leaves no doubt that an effective transdermal sodium hyaluronate complex, especially one as exemplified by the polymer matrix, would be an important modifier and facilitator in fast and uncomplicated healing following invasive surgical procedures, especially those procedures, which require stapling or suturing.

In addition, the same rationale can be applied to wounds of a long-standing nature where the natural biological components including the extracellular matrix have been compromised as in slow healing diabetic ulcers and bed sores.

The thrust of the current experimental work is to examine the compatibility of the components used for the construction of the bandage material, such as bandage products manufactured by Avery Dennison, for example, the Avery Dennison Web, with those of typical matrices used for various products. A matrix was formulated to serve a s prototype polymer matrix which would be suitable for the prevention of adhesions and scarring when impregnated or otherwise combined with the Web bandage.

Experiments to Examine Compatibility

Various matrices, including Diclofenac Matrix and Eczema Matrix, produced by the process of Example 1, were placed directly on the surface of the Avery Dennison Web. Various methods were used to dry the matrix including direct heat, infra-red heat and room temperature (3-5 minutes).

Once dry, the Web with matrix film was subjected to a variety of experiments. These included boiling, heating and otherwise using extreme adverse conditions to produce deterioration and destruction of the Web/Matrix combination material. The results of these experiments show that heat did not adversely affect the inherent qualities of the Web/Matrix, that is by hearing, with forced heat through a blower, or ultra-violet heat. The adhesive properties of the Avery Dennison Web are not reduced or otherwise compromised by the addition of polymer matrix.

Removing the matrix film produced no significant change in the adhesive qualities of the web bandaid material. In addition, the polymer matrices when applied directly to the padded portion of the Web/bandaid material absorb totally either when dried by the methods described above or when left for approximately 3-5 minutes on the surface of the padded portion at room temperature.

Description of Experiments and Results

A pro-forma wound healing matrix was manufactured using the process of Example 1. The formula was a follows:

Sodium Hyaluronate (NAHA) 2.5%

Hydroxyethylcellulose (HEC low weight) 156

Methoxyhydroxypolyethylene glycol (MPEG) 10%

Sodium D-Pantothenate 1.5%

Water q.s.

It was determined that the pro-forma wound healing matrix produced a film when heat was applied to the bandaid-type pad (Large Water Block Plus) which has a padded area of 2.5×5 cm. The heat method used was forced air drying or UV light. When the matrix was applied to the pad alone and left for 3-5 minutes at-room temperature, it dried and was integrated into the pad without producing a film. This method has several advantages: the integrated padded portion does not need moisture for rehydration, the material is active immediately when placed on the skin, and the cover for the bandaids and keep it dry.

Experiments with Niacin Matrix

In order to assess the transdermal activity associated with Web impregnated matrix, experiments were performed with a Niacin Matrix produced according to the procedures set forth above. The Niacin Matrix produces peripheral vasodilation with resulting redness, in a few minutes, at the site to which it is applied. Heat, at the site is also generated. These effects generally last for 15-20 minutes.

The experimental procedure described above was conducted using the large Bandaid-type: Large Water Block Plus with a useful area of 2.5×5 cm. All samples were dried at room temperature for 3-5 minutes and the gel was completely absorbed throughout the patch. The gel was applied carefully with a fine syringe and spread with a spatula. After applying the gel, the active area was sealed by pressing down the paper type (material covering the bandaid) on the four sides of the bandaid where the adhesive is located.

Testing Results

0.5 ml of Niacin Matrix was applied to the patch portion of the bandaid and was placed on the left arm of a subject and left in place for half-an-hour. The result was that reddening of the skin on the patch area was evident after the bandaid was removed.

In the second experiment, 0.75 ml of Niacin Matrix was applied, using similar technique to the patch. The patch was placed on the subject's right arm and was left in place for half-an-hour. There was increased reddening of the skin which radiated about 1 inch outward.

In the fourth experiment, 1.5 ml of Niacin gel was applied to the patch. However this was to much matrix, for the area, and the patch could not be successfully applied to the subject's skin.

Comments and Observations

There is a definite compatibility of the polymer matrix with bandaid (web material) either on the web itself or more successfully on the padded portion of the bandaid. The Matrix appears to be very effective when used on the Web and allowed to integrate by room temperature air drying for 3-5minutes.

Conclusions

From the preliminary experiments conducted to date, it would appear that a number of presentations can be developed using the Web/Matrix technology. Ultimately the objective would be to impregnate or coat the covering to be applied to the skin with an appropriate amount of matrix. The material covering the patch following matrix impregnation may be of the Teflon coated variety to avoid interaction or contact, or be made of any other suitably approved polymer material generally used for medical applications. Such materials should remain inert or non reactive with the matrix material.

The design and shape of the wound healing product will depend on the nature of the wound to be dressed. Obviously the length is determined again by the length of the wound itself. When treating ulcerated wounds which occur through illness or a deficiency, as in the case of bed sores, circular designs may work well.

Another factor that would appear to be favorable is the ability of the health care professional treating the patient or the patient themselves, to use additional matrix, where necessary, to heal stubborn conditions such as diabetic or slow healing ulcers. This would appear to be able to be accomplished simply by adding additional matrix to the already impregnated product at the time of application.

The matrix can also be presented in individual dispensing cartridges containing an appropriate amount of product. Such single dose cartridges are available through several sources. The best we feel is Confab in Quebec. The use of these cartridges may be customized for polymer matrix formulations and could be used to provide additional matrix for application to Web/pad materials in the treatment of difficult cases.

The following examples are illustrative of preferred embodiments of the invention and are not to be construed as limiting the invention thereto. All polymer molecular weights are mean average molecular weights. All percentages are based on the percent by weight of the final delivery system or formulation prepared unless otherwise indicated and all totals equal 100% by weight.

The following is a description of an embodiment of the dermal adhesive dressing of the present invention. This dressing is illustrated in FIG. 1 of the drawings. The adhesive dressing comprises a backing sheet 12 having apertures therein, a polymer matrix 15 and a porous covering layer 24. The upper surface of the backing sheet was coated with a layer of a pressure sensitive adhesive 14. It will be understood that any of the adhesive well known in the art for use with adhesive bandages may be used in place of this adhesive. The adhesive may, if desired, be deposited on the backing sheet in a continuous or discontinuous pattern rather than as an overall coating, as seen in the drawing.

The upper surface of the backing sheet carries and has adhered thereto a polymer matrix 15. The polymer matrix 15 is centered from end-to-end of the backing sheet and extends from side of the backing sheet to the other (see FIG. 1). The upper surface of the polymer matrix 15 is covered by a webbed covering material 24. Other porous covering materials may be used in place of the aforementioned polyethylene film.

The polymer matrix 15 used in the adhesive dressing of this example contains a supersaturated solution of hyaluronic acid. The webbed covering layer 24 overlies the upper surface of the polymer matrix and is coextensive in length and width with the polymer matrix.

FIG. 2 shows the above dressing with the addition of a support substrate 13. The support substrate 13 is preferably provided in the form of a fibrous pad which is centered from end-to-end of the backing sheet 12 and extends from one side of the backing sheet to the other. It will be understood that the support substrate 13 is secured to the backing sheet 12 by the aforementioned adhesive layer 14. The polymer matrix 15 overlies the support substrate 13. The function of the support substrate is to support the polymer matrix, as well as to provide a desirable cushioning effect when the adhesive dressing is applied over a wound site. The upper surface of the polymer matrix 15 may be covered by a webbed covering layer 24, as discusses above.

FIGS. 3 and 4 show different views of the dressing of FIG. 2 with the release sheets 18, 20 shown. Release sheets 18, 20 were placed over the exposed portions of adhesive 14 and the upper surface of the webbed covering layer 24 in such as way as to create tabs. The tabs are used to remove the release sheets before administering the dressing.

In addition, FIG. 4 shows pores 26 in the webbed covering layer 24. When the dressing is applied to a wound, the pressure will force drug in the polymer matrix up through the pores 26, allowing the drug to contact the skin.

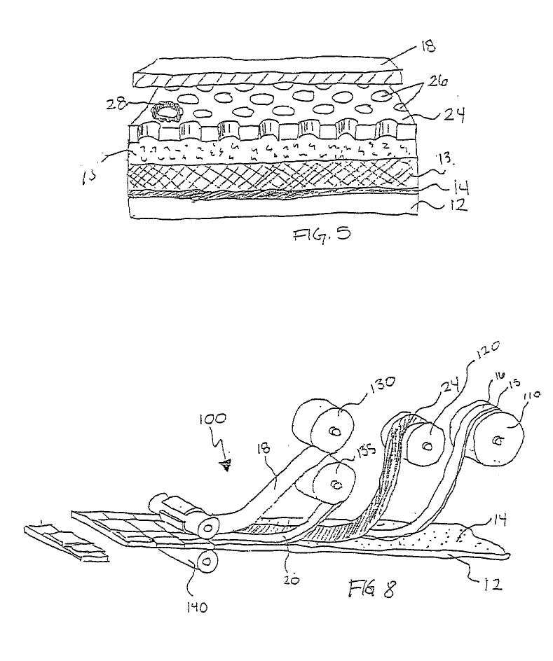

FIG. 5 shows a sectional view of the dressing of FIG. 2, allowing a view of all of the layers of the present invention. The upper surface of the backing sheet 12 is coated with an adhesive layer 14. The support substrate 15 rests upon the adhesive layer. The polymer matrix 15 overlies the support substrate 16 and the covering layer 24 overlies the polymer matrix 15. Pores 26 in the covering layer 24 are shown. In addition, the figure shows polymer matrix containing the drug 28, which has been forced through the pores 26 and is now able to contact the skin when applied.

FIG. 6 is a perspective view of another embodiment of the invention with portions cut away. The device comprises a laminated composite of adhesive overlay 68, a backing sheet 65 underlying adhesive overlay 68 and a membrane 70 permeable to the polymer matrix contained within reservoir 55. Release sheets 67 cover the adhesive. A support structure 60 may also be included.

The reservoir 55 is admixed to either support structure 60 or backing sheet 65. The reservoir may be admixed by gluing, mechanically fixing or through interlocking means or any other means known to one of ordinary skill. Alternatively, the reservoir 55 may be molded or integrally formed from the material forming the backing sheet or the support structure.

A peel disc (not shown) may underlie the permeable membrane and a heat seal (not shown) may be set about the periphery of the peel seal disc. The peel seal disc protects against release of the active agent from the reservoir and the heat seal protects the active agent from exposure to the environment prior to use.

Finally, the permeable membrane may contain apertures 71 to facilitate delivery of the polymer matrix. Alternatively, the membrane may be impermeable with the apertures 71 being the only means of delivery of the polymer matrix, wherein the apertures are configured to control the delivery and release rate of the polymer matrix.

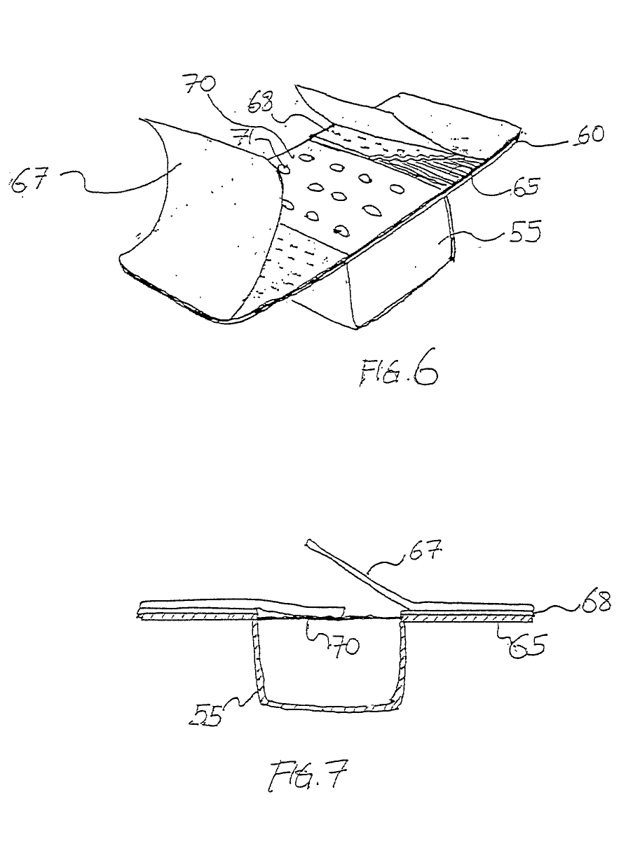

FIG. 7 is a cross-sectional view of the dressing of FIG. 6, showing the reservoir integrally formed or molded with the backing sheet 65.

Specifically, the adhesive dressing of FIG. 2 is manufactured according to a process in which the dressing is oriented at right angles to the direction of travel of the raw materials through the manufacturing apparatus. The backing sheet 12 coated with adhesive 14 is conveyed, from right to left, on top of a conveyor belt (not pictured) As shown in FIG. 5, a web comprising the support substrate 15 onto which the polymer matrix 16 has been previously applied by an extrusion coating process is led off of the roll 110 and placed on top of the adhesive 14 coated backing sheet 12. The width of the web corresponds to the length of the backing sheet.

The covering layer 24 is led off a supply roll 120 and placed on top of the web. It will be understood that in the process being described, the width of the covering layer corresponds substantially to the width of the web. Release sheets 18, 20, taken from rolls 130, 135, are applied so as to cover the exposed adhesive area at the other side of the adhesive coated backing sheet 12 as well as the upper surface of covering layer 24. Release sheets 18,20 extend beyond the edge of the covering layer 24 to provide grasping tabs.

The combined raw materials, as described above, are then passed through the nip of cutter rollers 140. The rollers compress the raw materials at a pressure of about 10-20 pounds per square inch and, at the same time, cut the traveling raw materials into individual adhesive dressings. As a result of the described process, the polymer matrix 16 is pressed up into holes 26, 28 in the covering layer 24 so that the polymer matrix is in intimate contact with the lower surfaces of release sheets 18, 20, as shown in FIG. 4. The individual adhesive dressings are subsequently wrapped, sterilized and packaged according to procedures which are well known in the art.

The adhesive dressing of the present invention can be applied to various portions of the skin of an animal in need of such treatment. A non-limiting list of examples of body parts for which the present adhesive dressing is useful includes the forehead, nose, neck, throat, arm, elbow, wrist, finger, chest, stomach, back, breast, leg, knee, ankle, foot and toe. In order to best fit specific body parts, the dressing of the present invention may be rectangular, as shown in FIG. 1. In additional embodiments of the present invention, the dressing may be circular or butterfly shaped (“H” shaped to best fit around fingers and toes). The dressing of the present invention can be small, large or sized to fit a specific body part.

The adhesive dressing of the present invention may also be in the form of a patch. The patch may be placed against the skin to administer a dosage of hyaluronic acid or its salts, alone or in combination with another drug, to an animal. The patch may be placed anywhere on the body where there is skin. Preferably, the patch may be placed on the back of the neck.

### EXAMPLE 1

This example illustrates the synthesis of a composition of the present invention. The following ingredients are combined as indicated.

| Ingredient | Quantity (grams) |
|---|---|
| Hyaluronate Sodium (HA) | 13.7 |
| Sterile Water | 900 |

Into a sterilized glass vessel is added 500 ml of the sterile water which is stirred at 400-600 rpms. Slowly add 13.7 grams of HA having an average molecular weight of around 700,000 to 775,000.

Allow to stir for 10 to 20 hours until all the HA has dissolved into the water and a crystal clear viscous solution has formed.

A quantity (500 grams) of the above viscous solution is placed in a clean beaker of known weight. A magnetic stirrer of known weight is placed in the beaker. The beaker containing the viscous solution and the stirrer is placed in a laboratory hood where the beaker and its contents are maintained in a warm location at 40° C. while being constantly stirred. Under these conditions water is removed from the viscous solution without any molecular degradation of the HA. At the end of one hour the beaker is weighed. If the weight reduction does not indicate removal of the desired amount of water, the beaker, with its contents, is returned to the warm location in the hood for further water removal.

In this example removal of 37 weight percent of the water is deemed sufficient to prepare a semi-solid material.

### EXAMPLE 2

This example illustrates the synthesis of a composition of the present invention employing hydroxyethylcellulose (HEC) as a nonionic polymer. The following ingredients are combined as indicated.

| Ingredient | Quantity (grams) |
|---|---|
| Hydroxyethylcellulose (HEC) | 12.5 |
| Hyaluronate Sodium (HA) | 13.7 |
| Sterile Water | 900 |

Into a sterilized glass vessel is added 500 ml of the sterile water which is stirred at 400-600 rpms. Slowly add 13.7 grams of HA having an average molecular weight of around 700,000 to 775,000 and a purity described previously.

Allow to stir for 10 to 20 hours until all the HA has dissolved into the water and a crystal clear viscous solution has formed.

Prepare a 1.25% solution of HEC by adding 12.5 grams of the solid material under aseptic conditions to 275 ml of sterile water. Allow to dissolve for 1 to 2 hours while stirring thereby forming an HEC solution. Add the HEC solution to the HA solution and mix until a homogenous clear viscous solution which is produced.

A quantity (500 grams) of the above viscous solution is placed in a clean beaker of known weight. A magnetic stirrer of known weight is placed in the beaker. The beaker containing the viscous solution and the stirrer is placed in a laboratory hood where the beaker and its contents are maintained in a warm location at 40° C. while being constantly stirred. Under these conditions water is removed from the viscous solution without any molecular degradation of the HA. At the end of one hour the beaker is weighed. If the weight reduction does not indicate removal of the desired amount of water, the beaker, with its contents, is returned to the warm location in the hood for further water removal.

In this example removal of 68 weight percent of the water is deemed sufficient to prepare a semi-solid sodium hyaluronate polymer matrix delivery system.

### EXAMPLE 3

This example demonstrates the formation of a transdermal nonsteroidal anti-inflammatory preparation known as diclofenac which produces relief of osteoarthritic and associated pain in areas affected by the disease. Such areas include, but are not limited to, knees, ankles, feet, back, neck, elbows, and hips.

The present example also demonstrates the formation of a transdermal preparation containing the NSAID drug when administered topically to sites affected by rheumatic or osteoarthritic disease will have an analgesic and beneficial effect. The onset of this beneficial effect in the form of pain relief and reduction of inflammation occurs between 10 and 20 minutes following topical administration and lasts for up to 6 hours.

The dosage range for the drug is between 2-4 ml (60 mg-120 mg) depending on the severity and site of the affected area.

Material

Diclofenac sodium 3%

Sodium hyaluronate (HA) 2.3%

Hydroxyethyl cellulose (HEC) 0.7%

Methoxypolyethylene glycol (MPEG) 10%

Benzyl alcohol 2.5%

Water Remainder

Batch Size 1500 ml

Into a sterilized glass vessel is added 1062.5 ml of sterile water which is stirred at 1500 to 2000 rpm. Slowly add 34.5 grams of HA, having a molecular weight of around 700,000 to 775,000 and a purity described above. Allow to stir for 16 to 20 hours until all of the HA polymer has dissolved into the water and a crystal-clear viscous solution has formed.

Prepare a 0.7% solution of HEC by adding 10.5 grams of the solid material under aseptic conditions to 250 ml of sterile water. Allow to dissolve for 1 to 2 hours while stirring at 1500 to 2000 rpm. Add the HEC solution to the HA solution and mix for 10 to 15 hours until a homogeneous solution is produced.

Carefully measure 150 ml of methoxypolyethylene glycol (MPEG) 10% into the mixture. RPM speeds should be increased for the mixture while this step is being performed to 2500 rpm. The resulting mixture thus formed should be allowed to mix at 2000 rpm for an additional 3 to 4 hours.

At this point 2.5% of benzol alcohol or 37.5 ml is added to the mixture. Again, the rpm speed is increased during this part of the procedure to 2500. The mixture should be allowed to mix for 3 to 5 hours at 2000 rpm.

Using safe techniques, 45 grams (3%) of the diclofenac should be slowly added to the mixture. Again the rpm speed for the purpose of addition of diclofenac should be increased to 2500, and the entire 45 grams of diclofenac should be completed within 15 minutes.

The final mixture is clear with a slight green tint following 15 to 20 hours of further mixing at 2000 rpm. The final product should be transferred, using aseptic technics, to 25 ml borasylicate glass jars with a lined cap.

### EXAMPLE 4

The formula and method of manufacture of Example 3 are repeated for diclofenac potassium. The only difference is that MPEG is not used.

Materials

Diclofenac potassium 3%

Sodium hyaluronate (HA) 2.3%

Hydroxyethyl cellulose (HEC) 0.7%

Benzyl alcohol 2.5%

BATCH SIZE 1500 ml

Into a sterilized glass vessel is added 1062.5 ml of sterile water which is stirred at 1500 to 2000 rpm. Slowly add 34.5 grams of HA, having a molecular weight of around 700,000 to 775,000 and a purity described previously. Allow to stir for 16 to 20 hours until all of the HA polymer has dissolved into the water and a crystal-clear viscous solution has formed.

Prepare a 0.7% of HEC by adding 10.5 grams of the solid material under aseptic conditions to 250 ml of sterile water. Allow to dissolve for 1 to 2 hours while stirring at 1500 to 2000 rpm. Add the HEC solution to the HA solution and mix for 10 to 15 hours until a homogeneous solution is produced.

At this point 2.5% of benzol alcohol or 37.5 ml is added to the mixture. Again, the rpm speed is increased during this part of the procedure to 2500. The mixture should be allowed to mix for 3 to 5 hours at 2000 rpm.

As described above, using safe techniques, 45 grams (3%) of the diclofenac is slowly added to the mixture. Again the rpm speed for the purpose of addition of diclofenac should be increased to 2500, and the entire 45 grams of diclofenac should be completed within 15 minutes.

The final mixture is clear with a slight green tint following 15 to 20 hours of further mixing at 2000 rpm. The final product should be transferred, using aseptic technic, to 25 ml borasylicate glass jars with a lined cap.

### EXAMPLE 5

The general manufacturing procedure of Example 3 is repeated for a topical dermalogical preparation. The main difference in composition is the use of methylparabin as a preservative.

Materials

Sodium hyaluronate (HA) 2.5%

Hydroxyethyl cellulose (HEC) 1.25%

Benzyl alcohol 1%

Methyl parabin 0.2%

Water Q.S.

Prior to dissolving the HA into the water, methyl parabin is dissolved and then HA added thereto. The remaining process steps of Example 1 were then repeated.

When 3 to 5 milliliters of this formulation was applied to pressure sores 3 to 4 times daily, the tissue healed and returned to a normal condition within 4 to 7 days.

### EXAMPLE 6

The following describes experiments with respect to a pro-forma wound healing dressing. The formula was as follows:

| | |
|---|---|
| Sodium Hyaluronate (NAHA) | 2.5% |
| Hydroxyelthylcellulose (HEC low weight) | 156 |
| Mthooxyhydroxypolyethylene glycol (MPEG) | 10% |
| Sodium D-Pantothenate | 1.5% |
| Water g.s. | |

It was determined that the pro-forma wound healing matrix produced a sheet or film when heat was applied to the mixture. The heat method used was force air drying or ultraviolet light. When the wound healing matrix was poured into a petri dish to the depth of 0.2 inches and left for 30 minutes at room temperature the same sheet was produced. This method has several advantages: the sheet does not require rehydration for treatment purposes, the sheet is immediately active when placed on the skin and the sheet is easily protectable by a gauze or the type of dressing fixative material.

### EXAMPLE 7

This example illustrates the synthesis of a composition of the present invention employing hydroxyethyl cellulose (HEC) as a nonionic polymer in the polymer matrix.

The following ingredients are combined as indicated.

| | Quantity |
|---|---|
| Ingredient | (grams) |
| Hydroxyethyl cellulose (HEC) | 12.5 |
| Hyaluronate Sodium (HA) | 13.7 |
| Sterile Water | 900 |

Into a sterilized glass vessel is added 500 ml of the sterile water which is stirred at 400-600 rpms. Slowly add 13.7 grams of HA having a molecular weight of around 700,000 to 775,000 and a purity described previously.

Allow to stir for 10 to 20 hours until all the HA has dissolved into the water and a crystal clear viscous solution has formed.

Prepare a 1.25% solution of HEC by adding 12.5 grams of the solid material under aseptic conditions to 275 ml of sterile water. Allow to dissolve for 1 to 2 hours while stirring thereby forming an HEC solution. Add the HEC solution to the HA solution and mix for 2 to 4 hours at 490 to 600 rpm until a homogenous clear viscous solution which is free of air bubbles is produced.

A quantity (500 grams) of the above viscous solution is placed in a clean beaker of known weight. A magnetic stirrer of known weight is placed in the beaker. The beaker containing the viscous solution and the stirrer is placed in a laboratory hood where the beaker and its contents are maintained in a warm location at 40° C. while being constantly stirred. Under these conditions water is removed from the viscous solution without any molecular degradation of the HA. At the end of one hour the beaker is weighed. If the weight reduction does not indicate removal of the desired amount of water, the beaker, with its contents, is returned to the warm location in the hood for further water removal.

### EXAMPLE 8

This example demonstrates the formation of a transdermal nonsteroidal anti-inflammatory preparation of the matrix utilizing diclofenac, which produces relief of osteoarthritic and associated pain in areas affected by the disease. Such areas include, but are not limited to, knees, ankles, feet, back, neck, elbows, and hips. The present example also demonstrates the formation of a transdermal preparation containing the NSAID drug which when administered topically to sites affected by rheumatic or osteoarthritic disease will have an analgesic and beneficial effect. The onset of this beneficial effect in the form of pain relief and reduction of inflammation occurs between 10 and 20 minutes following topical administration and lasts for up to 6 hours.

The dosage range for the drug is between 2-4 ml (60 mg-120 mg) depending on the severity and site of the affected area.

Ingredient

Diclofenac sodium 3%

Sodium hyaluronate (HA) 2.3%

Hydroxyethyl cellulose (HEC) 0.7%

Methoxypolyethylene glycol (MPEG) 10%

Benzyl alcohol 2.5%

Water Remainder

Into a sterilized glass vessel is added 1062.5 ml of sterile water which is stirred at 1500 to 2000 rpm. Slowly add 34.5 grams of HA, having an average molecular weight of around 700,000 to 775,000 and a purity described above. Allow to stir for 16 to 20 hours until all of the HA polymer has dissolved into the water and a clystal-clear viscous solution has formed.

Prepare a 0.7% solution of HEC by adding 10.5 grams of the solid material under aseptic conditions to 250 ml of sterile water. Allow to dissolve for 1 to 2 hours while stirring at 1500 to 2000 rpm. Add the HEC solution to the HA solution and mix for 10 to 15 hours until a homogeneous solution is produced.

Carefully measure 150 ml of methoxypolyethylene glycol (MPEG) 10% into the mixture. RPM speeds should be increased to 2500 rpm for the mixture while this step is being performed. The resulting mixture thus formed should be allowed to mix at 2000 rpm for an additional 3 to 4 hours.

At this point 2.5% of benzol alcohol or 37.5 ml is added to the mixture. Again, the rpm speed is increased during this part of the procedure to 2500 rpm. The mixture should be allowed to mix for 3 to 5 hours at 2000 rpm.

Using safe techniques, 45 grams (3%) of the diclofenac should be slowly added to the mixture. Again the rpm speed for the purpose of addition of diclofenac should be increased to 2500, and the entire 45 grams of diclofenac should be completed within 15 minutes.

The final mixture is clear with a slight green tint following 15 to 20 hours of further mixing at 2000 rpm. The final product should be transferred, using aseptic techniques, to 25 ml borasylicate glass jars with a lined cap.

### EXAMPLE 9

The following describes experiments with respect to a pro-forma wound healing dressing. The formula was as follows:

| | |
|---|---|
| Sodium Hyaluronate (NAHA) | 2.5% |
| Hydroxyelthylcellulose (HEC low weight) | 1.56% |
| Mthooxyhydroxypolyethylene glycol (MPEG) | 10% |
| Sodium D-Pantothenate | 1.5% |
| Water g.s. | |

It was determined that the pro-forma wound healing matrix produced a sheet or film when heat was applied to the mixture. The heat method used was force air drying or ultraviolet light. When the wound healing matrix was poured into a petri dish to the depth of 0.2 inches and left for 30 minutes at room temperature the same sheet was produced. This method has several advantages: the sheet does not require rehydration for treatment purposes, the sheet is immediately active when placed on the skin. Additionally, the sheet is easily protectable by a gauze or the type of dressing fixative material.

The invention being thus described, it will be obvious that the same may be varied in many ways. Such variations are not to be regarded as a departure from the spirit and scope of the invention and all such modifications are intended to be included within the scope of the following claims.

What is claimed is:

1. A stable, sterilized, concentrated polymer matrix, comprising:
a negatively charged polymer material which may be selected from the group consisting of polysulfated glucosoglycans, glycosaminoglycans, mucopolysaccharides and mixtures thereof; and
a nonionic polymer which may be selected from the group consisting of carboxymethylcellulose sodium, hydroxyethyl cellulose, hydroxypropyl celluose, and mixtures thereof.

2. The concentrated polymer matrix of claim 1, wherein a drug can be combined with said polymer matrix.

3. The concentrated polymer matrix of claim 1, wherein a molar ratio of negatively charged polymer to non-ionic polymer is 1:0.5 to 3.0.

4. The concentrated polymer matrix of claim 1, wherein a molar ratio of negatively charged polymer to non-ionic polymer is 1:0.7 to 2.5.

5. The concentrated polymer matrix of claim 1, wherein said negatively charged polymer is selected from the group of hyaluronic acid, a sodium or potassium salt of hyaluronic acid and mixtures thereof.

6. The concentrated polymer matrix of claim 1, wherein said composition is capable of continuously releasing therapeutically effective amounts of said drug over about 1 hour to about 24 hours of time when administered to an animal.

7. The concentrated polymer matrix of claim 1, wherein the polymer matrix is a formable, flexible, movable sheet.

8. The concentrated polymer matrix of claim 5, is wherein the hyaluronic acid is concentrated.

9. A dermal dressing, comprising:
a concentrated polymer matrix containing a negatively charged polymer in combination with a nonionic polymer, wherein the polymer matrix is conformable to topical application on animal skin

10. The dermal dressing of claim 9, wherein said polymer matrix contains a therapeutically effective amount of a drug.

11. The dermal dressing of claim 9, wherein said nonionic polymer is selected from the group consisting of hydroxyethylcellulose, hydroxypropylcellulose, or carboxymethylcellulose.

12. The dermal dressing of claim 9, wherein said negatively charged polymer is selected from the group of a hyaluronic acid, a hyaluronic acid salt and mixtures thereof.

13. The dermal dressing of claim 9, further comprising:
a backing sheet having applied thereto an adhesive capable of securing the dermal dressing to the animal skin;
a reservoir affixed to said backing sheet containing said polymer matrix; and
an inert porous membrane interposed between said polymer matrix and said animal skin.

14. The dermal dressing of claim 13, wherein the dermal dressing has a perimeter edge defining a circumference, a rectilinear perimeter, a triangular perimeter or a perimeter of any geometric shape.

15. The dermal dressing of claim 13, wherein the inert porous membrane has a delivery rate regulating means for dosing the drug over a period of time.

16. The dermal dressing of claim 15, wherein the delivery rate of the porous membrane is about 1 hour to about 24 hours per dose.

17. The dermal dressing of claim 13, further comprising:
a backing sheet overlying said polymer matrix, wherein the backing sheet having applied thereto an adhesive capable of securing the polymer matrix to the backing sheet and the backing sheet to the animal skin; and
a webbed covering layer underlying said polymer matrix.

18. The dermal dressing of claim 13, further comprising:
a covering layer overlying the polymer matrix;
one or more release sheets, wherein said backing sheet having applied thereto an adhesive which secures said support substrate to the backing sheet and the backing sheet to the animal skin;
wherein the release sheets completely cover the adhesive on the backing sheet and the covering layer; and
wherein the release sheets may be peeled off of said adhesive.

19. The dressing of claim 13, wherein the backing sheet is permeable to oil or water.

20. The dressing of claim 13, wherein the backing sheet is impermeable to oil or water.

21. The dressing of claim 13, wherein the backing sheet is inert to hyaluronic acid and its salts.

22. The dressing of claim 13, wherein the webbed covering layer is a natural polymer.

23. The dressing of claim 13, wherein the webbed covering layer is a synthetic polymer.

24. The dressing of claim 23, wherein the synthetic polymer is selected from the group consisting of polyvinyl chloride, polyethylene, polypropylene, polyester and nylon.

25. The dressing of claim 23, wherein the webbed covering layer is sufficiently porous to enable the polymer matrix to contact the skin.

26. A method for administering a drug to an animal, comprising the step of:
applying a dermal dressing to animal skin, wherein the dermal dressing is comprised of:
a concentrated polymer matrix containing a negatively charged polymer in combination with a nonionic polymer, wherein the polymer matrix is conformable to topical application on animal skin.

27. The method of claim 26 wherein, said concentrated polymer matrix contains a therapeutically effective amount of a drug.

28. A method for preventing or treating a condition in an animal comprising the steps of:
applying a concentrated polymer matrix film onto the animal on an area to be treated, wherein the concentrated polymer matrix film contains a negatively charged polymer in combination with a nonionic polymer, and is formable, flexible and moveable; and
securing said polymer matrix film onto the area to be treated with a dressing fixative.

29. The method of claim 28, wherein the dressing fixative is a bandage selected from the group consisting of a single sided adhesive bandage, a gauze wrap, a stretchable woven wrap and a stretchable sleeve.

30. The method of claim 28, wherein the polymer matrix film delivers a therapeutically effective amount of a drug upon the animal for about 1 hour to about 24 hours of time.

31. A method for preventing or treating a condition in an animal for a sustained period of time, comprising the step of:
applying to said animal a concentrated polymer matrix, comprising a negatively charged polymer and a nonionic polymer in combination with a therapeutically effective amount of a drug for preventing or treating nausea.
